(19) United States
(12) Patent Application Publication
(10) Pub. No.: US 2023/0158055 A1
(43) Pub. Date: May 25, 2023

(54) METHODS AND REGIMENS FOR THE TREATMENT OF HEMATOLOGICAL CANCER

(71) Applicant: Biosight Ltd., Lod (IL)

(72) Inventors: Liat FLAISHON, Ramat Gan (IL); Ruth BEN YAKAR, Shoham (IL)

(73) Assignee: Biosight Ltd., Lod (IL)

(21) Appl. No.: 17/920,183

(22) PCT Filed: Apr. 22, 2021
(86) PCT No.: PCT/IL2021/050460

§ 371 Date: Oct. 20, 2022

Related U.S. Application Data
(60) Provisional application No. 63/014,187, filed on Apr. 23, 2020.

(51) Int. Cl. A61K 31/7068; A61K 31/635; A61P 35/02; A61K 45/06

(52) U.S. Cl. CPC A61K 31/7068; A61K 31/635; A61P 35/02; A61K 45/06

(57) ABSTRACT

The invention provides methods and uses for the treatment of hematological cancer in a subject in need thereof.

### BACKGROUND OF THE INVENTION

Acute myeloid leukemia (AML) is associated with poor outcome in older patients and in patients unfit for standard induction therapy. Cytarabine serves as the backbone of treatment for AML for decades, however, standard cytarabine therapy is associated with severe side effects, such as cerebellar toxicity, bone marrow suppression, and infections, leading to high treatment-related mortality rates.

Hence, while the incidence of AML increases with age, advanced age and comorbidities may preclude the administration of intensive induction therapy altogether due to its potential toxicities. The outcome in older patients who are unable to receive intensive chemotherapy remains insufficient, in spite of recent approvals of new therapeutics.

Aspacytarabine is a novel proprietary anti-metabolite. It is composed of cytarabine covalently bound to aspartic acid, acting as a pro-drug of cytarabine, enabling delivery of high cytarabine doses to hematological malignancy and disorder patients with lower systemic exposure to the free drug. Following its administration, aspacytarabine is cleaved to cytarabine, enabling effective killing of target cells and relative sparing of normal tissues. As such, aspacytarabine may serve as an ideal therapy for hematological malignancies and disorders, particularly for delivering higher doses of cytarabine with better safety profile compared to free cytarabine.

### SUMMARY OF THE INVENTION

The present invention provides a method of treating hematological cancer in a subject in need thereof, wherein said method comprises: at least one initial therapy course and at least one subsequent therapy course; wherein said at least one initial therapy course is administered until said subject is in remission; and said at least one subsequent therapy course is administered while said subject is in remission; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

In some embodiments, this invention provides a method of treating hematological cancer in a subject in need thereof, wherein said method comprises: at least one initial therapy course and at least one subsequent therapy course; wherein said at least one initial therapy course is administered until said subject is in remission; and said at least one subsequent therapy course is administered while said subject is in remission; and wherein the at least one initial therapy course comprises administering at least one anti-hematological cancer agent, and the at least one subsequent therapy course comprises administering a composition comprising (2S)-2-amino-4-[[1-[(2R,3S,4S,5R)-3,4-dihydroxy-5-(hydroxymethyl)oxolan-2-yl]-2 -oxopyrimidin-4-yl]amino]-4-oxobutanoic acid (aspacytarabine) or a pharmaceutically acceptable salt thereof.

In some embodiments, this invention provides a method of treating hematological cancer in a subject in need thereof, wherein said method comprises: at least one initial therapy course and at least one subsequent therapy course; wherein said at least one initial therapy course is administered until said subject is in remission; and said at least one subsequent therapy course is administered while said subject is in remission; and wherein the at least one initial therapy course comprises administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof; and the at least one subsequent therapy course comprises administering at least one anti-hematological cancer agent.

In some embodiments, the methods described herein are for the treatment of hematological cancer. In other embodiments, the hematological cancer is selected from Leukemia, Lymphoma, Multiple Myeloma, Myelodysplastic Syndrome (MDS), myeloproliferative neoplasm (MPN) and any combinations thereof. In other embodiments, the Leukemia is selected from Acute Myeloid Leukemia (AML), Acute Lymphoblastic Leukemia (ALL), Chronic Myeloid Leukemia (CML), Chronic Lymphoblastic Leukemia (CLL), and any combinations thereof. In other embodiments, the lymphoma is selected from Hodgkin's Lymphoma, Non-Hodgkin's Lymphoma (NHL), and any combinations thereof.

In some embodiments, the methods of this invention make use of at least one anti-hematological cancer agent. In other embodiments, the at least one anti-hematological cancer agent is selected from BCL2 inhibitor, hypomethylation agent (HMA), anti-metabolite, targeted therapy, immune therapy, CAR-T, and any combinations thereof. In another embodiments, the at least one anti-hematological cancer agent is selected from venetoclax, cytarabine, aspacytarabine, daunorubicin, idarubicin, cyclosphosphamide, dexamethasone, daunorubicin hydrochloride and cytarabine liposome, azacitidine, decitabine, glasdegib, sorafenib, midostaurin, ivosidenib, enasidenib, gemtuzumab ozogamicin, gilteritinib, magrolimab, cusatuzumab, CD47 inhibitor, APR-246, CART-123 and any combinations thereof.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiment, provided herein a method or a regimen comprising administering various anti-hematological cancer agents for treating hematological cancer in a subject in need thereof, wherein said method or regimen comprises: at least one initial therapy course and at least one subsequent therapy course; wherein said at least one initial therapy course is administered until said subject is in remission; and said at least one subsequent therapy course is administered while said subject is in remission; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

In some embodiment, provided herein a method or a regimen for treating Myelodysplastic Syndrome (MDS) in a subject in need thereof, wherein said method or regimen comprises: at least one initial therapy course and at least one subsequent therapy course; wherein said at least one initial therapy course is administered until said subject is in hematological improvement; and said at least one subsequent therapy course is administered while said subject is in hematological improvement; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

In some embodiments, the methods or regimens described herein comprise at least one initial therapy course. In other embodiments, the initial therapy comprises between 1-4 courses when a composition comprising aspacytarabine is being administered. In another embodiment, the initial therapy comprises between 1-2 courses when a composition comprising aspacytarabine is being administered. In another embodiment, the initial therapy comprises 1 course when a composition comprising aspacytarabine is being administered. In another embodiment, the initial therapy comprises 2 courses when a composition comprising aspacytarabine is being administered. In another embodiment, the initial therapy comprises 3 courses when a composition comprising aspacytarabine is being administered. In another embodiment, the initial therapy comprises 4 courses when a composition comprising aspacytarabine is being administered. In another embodiments, each course is the same or different in terms of the number of days each course is being conducted.

In some embodiments, the methods or regimens described herein comprise at least one subsequent therapy course. In other embodiments, the subsequent therapy comprises between 1-4 courses when a composition comprising aspacytarabine is being administered. In another embodiment, the subsequent therapy comprises between 1-2 courses when a composition comprising aspacytarabine is being administered. In another embodiment, the subsequent therapy comprises 1 course when a composition comprising aspacytarabine is being administered. In another embodiment, the subsequent therapy comprises 2 courses when a composition comprising aspacytarabine is being administered. In another embodiment, the subsequent therapy comprises 3 courses when a composition comprising aspacytarabine is being administered. In another embodiment, the subsequent therapy comprises 4 courses when a composition comprising aspacytarabine is being administered. In another embodiments, each course is the same or different in terms of the number of days each course is being conducted.

In some embodiments, the at least one initial therapy comprises number of courses until remission, wherein each course is the same or different.

In some embodiments, the at least one subsequent therapy comprises number of courses, wherein each course is the same or different. In some embodiments, the at least one subsequent therapy comprises number of courses until disease progression, wherein each course is the same or different.

In some embodiments, the methods or regimens described herein comprise at least one initial therapy course and at least one subsequent therapy course, wherein each therapy course optionally comprises an anti-hematological cancer agent in combination with a composition comprising aspacytarabine.

In some embodiments, the methods or regimens described herein comprise at least one initial therapy course, wherein the initial therapy course comprises administering at least one anti-hematological cancer agent. In another embodiment, the anti-hematological cancer agent is aspacytarabine. In another embodiment, the anti-hematological cancer agent is not aspacytarabine. In another embodiment, the initial therapy course comprises administering an anti-hematological cancer agent in combination with a composition comprising aspacytarabine.

In another embodiment, the initial therapy course comprises administering at least two anti-hematological cancer agents, wherein one of said anti-hematological cancer agents is aspacytarabine. In another embodiment, the initial therapy course comprises administering at least one anti-hematological cancer agent that is not aspacytarabine.

In another embodiment, the initial therapy course comprises administering at least two anti-hematological cancer agents, wherein at least one of the anti-hematological cancer agents is not aspacytarabine.

In some embodiments, the methods or regimens described herein comprise at least one subsequent therapy course, wherein the subsequent therapy course comprises administering at least one anti-hematological cancer agent. In another embodiment, the anti-hematological cancer agent is aspacytarabine. In another embodiment, the anti-hematological cancer agent is not aspacytarabine. In another embodiment, the subsequent therapy course comprises administering an anti-hematological cancer agent in combination with a composition comprising aspacytarabine.

In another embodiment, the subsequent therapy course comprises administering at least two anti-hematological cancer agents, wherein one of said anti-hematological cancer agent is aspacytarabine. In another embodiment, the subsequent therapy course comprises administering at least one anti-hematological cancer agent that is not aspacytarabine.

In another embodiment, the at least one subsequent therapy course comprises administering at least two anti-hematological cancer agents, wherein at least one of said anti-hematological cancer agent is not aspacytarabine.

In some embodiments, provided herein are methods or regimens of treating hematological cancer in a subject in need thereof, wherein said methods or regimens comprise at least one initial therapy course, and at least one subsequent therapy course; wherein said at least one initial therapy course is administered until said subject is in remission; and said at least one subsequent therapy course is administered while said subject is in remission. In other embodiments, the at least one initial therapy course comprises administering a composition comprising aspacytarabine and at least one additional anti-hematological cancer agent; and wherein said at least one subsequent therapy course comprises administering a composition comprising aspacytarabine and at least one additional anti-hematological cancer agent; wherein the at least one additional anti-hematological cancer agent in the initial and subsequent therapy is the same or different.

In some embodiments, provided herein are methods or regimens of treating MDS in a subject in need thereof, wherein said methods or regimens comprise at least one initial therapy course, and at least one subsequent therapy course; wherein said at least one initial therapy course is administered until said subject is in hematological improvement; and said at least one subsequent therapy course is administered while said subject is in hematological improvement. In other embodiments, the at least one initial therapy course comprises administering a composition comprising aspacytarabine and at least one additional anti-hematological cancer agent; and wherein said at least one subsequent therapy course comprises administering a composition comprising aspacytarabine and at least one additional anti-hematological cancer agent; wherein the at least one additional anti-hematological cancer agent in the initial and subsequent therapy is the same or different.

In some embodiments, provided herein are methods or regimens of treating hematological cancer in a subject in need thereof, wherein said methods or regimens comprise at least one initial therapy course, and at least one subsequent therapy course; wherein said at least one initial therapy course is administered until said subject is in remission; and said at least one subsequent therapy course is administered while said subject is in remission. In other embodiments, the at least one initial therapy course comprises administering a composition comprising aspacytarabine and at least one additional anti-hematological cancer agent; and wherein said at least one subsequent therapy course comprises a composition comprising aspacytarabine.

In some embodiments, provided herein are methods or regimens of treating MDS in a subject in need thereof, wherein said methods or regimens comprise at least one initial therapy course, and at least one subsequent therapy course; wherein said at least one initial therapy course is administered until said subject is in hematological improvement; and said at least one subsequent therapy course is administered while said subject is in hematological improvement. In other embodiments, the at least one initial therapy course comprises administering a composition comprising aspacytarabine and at least one additional anti-hematological cancer agent; and wherein said at least one subsequent therapy course comprises a composition comprising aspacytarabine.

In some embodiments, provided herein are methods or regimens of treating hematological cancer in a subject in need thereof, wherein said methods or regimens comprise at least one initial therapy course, and at least one subsequent therapy course; wherein said at least one initial therapy course is administered until said subject is in remission; and said at least one subsequent therapy course is administered while said subject is in remission. In other embodiments the at least one initial therapy course comprises administering at least one anti-hematological cancer agent other than aspacytarabine; and wherein said at least one subsequent therapy course comprises aspacytarabine and at least one additional anti-hematological cancer agent.

In some embodiments, provided herein are methods or regimens of treating MDS in a subject in need thereof, wherein said methods or regimens comprise at least one initial therapy course, and at least one subsequent therapy course; wherein said at least one initial therapy course is administered until said subject is in hematological improvement; and said at least one subsequent therapy course is administered while said subject is in hematological improvement. In other embodiments the at least one initial therapy course comprises administering at least one anti-hematological cancer agent other than aspacytarabine; and wherein said at least one subsequent therapy course comprises administering a composition comprising aspacytarabine and at least one additional anti-hematological cancer agent.

In some embodiments, provided herein are methods or regimens of treating hematological cancer in a subject in need thereof, wherein said methods or regimens comprise at least one initial therapy course, and at least one subsequent therapy course; wherein said at least one initial therapy course is administered until said subject is in remission; and said at least one subsequent therapy course is administered while said subject is in remission. In other embodiments, the at least one initial therapy comprises administering at least one anti-hematological cancer agent other than aspacytarabine; and wherein the at least subsequent therapy course comprises administering a composition comprising aspacytarabine.

In some embodiments, provided herein are methods or regimens of treating MDS in a subject in need thereof, wherein said methods or regimens comprise at least one initial therapy course, and at least one subsequent therapy course; wherein said at least one initial therapy course is administered until said subject is in hematological improvement; and said at least one subsequent therapy course is administered while said subject is in hematological improvement. In other embodiments, the at least one initial therapy comprises administering at least one anti-hematological cancer agent other than aspacytarabine; and wherein the at least subsequent therapy course comprises administering a composition comprising aspacytarabine.

In some embodiments, this invention provides a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof for use in the treatment of hematological cancer in a subject in need thereof, wherein said treatment comprises: at least one initial therapy course and at least one subsequent therapy course; wherein said at least one initial therapy course is administered until said subject is in remission; and wherein said at least one subsequent therapy course comprises administering said composition while said subject is in remission; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

In some embodiments, this invention provides a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof for use in the treatment of MDS in a subject in need thereof, wherein said treatment comprises: at least one initial therapy course and at least one subsequent therapy course; wherein said at least one initial therapy course is administered until said subject is in hematological improvement; and wherein said at least one subsequent therapy course comprises administering said composition while said subject is in hematological improvement; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

When referring to “hematological cancer” it should be understood to encompass tumors that affect the blood, bone marrow, lymph, and lymphatic system. Because these tissues are all intimately connected through both the circulatory system and the immune system, a disease affecting one will often affect the others as well, making myelo-proliferation and lympho-proliferation (and thus the leukemias and the lymphomas). This commonly leads to a different approach in diagnosis and treatment of hematological malignancies. Hematological malignancies are malignant neoplasms (“cancer”), and they are generally treated by specialists in hematology and/or oncology.

Typically, hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

In some embodiments, said hematological cancer refers to pre-cancerous condition. In another embodiment, the pre-cancerous condition is Myelodysplastic Syndrome (MDS).

In some embodiments, said hematological cancer is selected from Leukemia, Lymphoma, Multiple Myeloma, Myeloproliferative Neoplasm (NPM) and any combinations thereof. In another embodiment, said hematological cancer is Leukemia. In another embodiment, said hematological cancer is Lymphoma. In another embodiment, said hematological cancer is Multiple Myeloma. In another embodiment, said hematological cancer is Myeloproliferative Neoplasm (MPN).

In further embodiments, Lymphoma is selected from Hodgkin's Lymphoma, Non-Hodgkin's Lymphoma (NHL), and any combinations thereof.

In other embodiments, Leukemia is selected from Acute Myeloid Leukemia (AML), Acute Lymphoblastic Leukemia (ALL), Chronic Myeloid Leukemia (CML), Chronic Lymphoblastic Leukemia (CLL), and any combinations thereof. In another embodiment, said Leukemia is Acute Myeloid Leukemia (AML). In another embodiment, said Leukemia is Acute Lymphoblastic Leukemia (ALL). In another embodiment, said Leukemia is Chronic Myeloid Leukemia (CML). In another embodiment, said Leukemia is Chronic Lymphoblastic Leukemia (CLL).

In another embodiment, Acute Myeloid Leukemia (AML) refers to newly diagnosed acute myeloid leukemia (AML) subjects. In another embodiment, Acute Myeloid Leukemia (AML) refers to newly diagnosed acute myeloid leukemia (AML) subject which is unfit for intensive therapy. In another embodiment, Acute Myeloid Leukemia (AML) subject refers to an adult subject which is newly diagnosed with acute myeloid leukemia (AML) which is unfit for intensive therapy.

The term “intensive therapy” refers to high dose chemotherapy.

In another embodiment, Acute Myeloid Leukemia (AML) refers to relapse Acute Myeloid Leukemia (AML). In another embodiment, acute Myeloid Leukemia (AML) refers to refractory AML. In another embodiment, Acute Myeloid Leukemia (AML) refers to relapse refractory Acute Myeloid Leukemia (AML).

In another embodiment, provided herein is a treatment for a subject which is diagnosed with acute myeloid leukemia (AML) which is unfit for intensive therapy, wherein the treatment comprises up to two courses of initial therapy with a composition comprising aspacytarabine in combination with venetoclax followed by up to three courses of subsequent therapy with aspacytarabine.

In another embodiment, provided herein is a treatment for a subject which is newly diagnosed acute myeloid leukemia (AML) which is unfit for intensive therapy, wherein the treatment comprises up to two courses of initial therapy with a composition comprising aspacytarabine in combination with venetoclax followed by up to three courses of subsequent therapy with aspacytarabine.

In some embodiments, provided herein is a treatment for AML subjects in first remission following initial treatment of venetoclax and azacitidine, wherein the subjects are being treated for 2-4 subsequent therapy courses comprising aspacytarabine.

In another embodiment, provided herein a treatment for a subject which is unfit for intensive therapy.

When referring to an “initial therapy course” it should be understood to encompass the first phase of treatment that is aimed at achieving a disease remission and/or hematological improvement (in MDS subjects), removing as many cancerous cells as possible. After a predetermined time period or number of courses, the caretaker determines (according to, for example, bone marrow, blood, and/or other tests and parameters) if the cancer is considered in remission (either complete or partial remission, for example in leukemia, tests should show a decrease in bone marrow blasts). When a patient is in complete remission the blasts are making less than 5% of the bone marrow, while in partial remission, bone marrow blast percentage is reduced to between 5% to 25%; and decrease of pretreatment bone marrow blast percentage by at least 50%. For hematological improvement in MDS patients, there should be an improvement in blood cells count (Hgb increase by 1.5 g/dl, Platelets absolute increase of ≥30×109/L if starting with >20×10⁹/L platelets or increase from <20×10⁹/L to >20×10⁹/L and by at least 100%, neutrophil increase of at least 100% and an absolute increase >0.5×10⁹/L.

In the context of the present invention, said initial therapy course comprises, optionally among others, administration of a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof. Initial therapy course is administered to said subject until the subject is in remission. Initial therapy course is administered to said subject until the subject is in hematological improvement.

A partial and/or complete remission definition is as disclosed in Döhner, Hartmut, et al, “Diagnosis and management of AML in adults: 2017 ELN recommendations from an international expert panel.” Blood 129.4 (2017): 424-447, which is incorporated herein by reference.

Hematological improvement is defined according to the international working group definition as disclosed in Cheson, Bruce D., et al. “Clinical application and proposal for modification of the International Working Group (IWG) response criteria in myelodysplasia. Blood, 108.2 (2006). 419-425, as incorporated herein by reference.

In MDS subjects the hematological improvement (HI) according to the international working group refers to a specific response of cytopenias in three hematopoietic lineages: erythroid (HI-E), platelet (HI-P), and neutrophil (HI-N):

a. Erythroid response (pretreatment, <11 g/dL) is defined as Hgb increase by ≥1.5 g/dL. Relevant reduction of units of red blood cells (RBC) transfusions by an absolute number of at least 4 RBC transfusions/8 weeks, compared with the pretreatment transfusion number in the previous 8 weeks. Only RBC transfusions given for a Hgb of ≤9.0 g/dL will count in the RBC transfusion response evaluation.
b. Platelet response (pretreatment <100×10⁹/L) is defined as absolute increase of ≥30×10⁹/L for patients starting with >20×10⁹/L platelets, and an increase from <20×10⁹/L to >20×10⁹/L and by at least 100%.
c. Neutrophil response (pretreatment, <1.0×10⁹/L) is defined as at least 100% increase and an absolute increase >0.5×10⁹/L.

The HIs are measured in patients with pretreatment abnormal values: hemoglobin level less than 110 g/L (11 g/dL) or RBC-transfusion dependence, platelet count less than 100×10⁹/L or platelet-transfusion dependence, absolute neutrophilcount (ANC) less than 1.0×10⁹/L. Pretreatment baseline measures of cytopenias are averages of at least 2 measurements (not influenced by transfusions, i.e., no RBC transfusions for at least 1 week and no platelet transfusions for at least 3 days) over at least 1 week prior to therapy.

When referring to said at least one initial therapy course being “administered until said subject is in remission” it should be understood to mean that the at least one initial therapy course is administered to said subject until the parameters of said subject show that the cancer the subject is suffering from is in remission (any type of remission including but not limited to complete remission, complete remission with incomplete hematological recovery, partial remission, and so forth).

When referring to an “subsequent therapy course” (in-remission therapy course, or in hematological improvement therapy in MDS subjects,) it should be understood to encompass the second phase of treatment, following signs of remission in said subject, that includes further chemotherapy treatment to aim at destroying any remaining cancer cells and help prevent a relapse. In the context of the present invention, said subsequent therapy course comprises, optionally among others, administration of a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine). Subsequent therapy course is administered to said subject while said subject is in remission. Subsequent therapy course is administered to said subject while said subject is in hematological improvement.

In some embodiments, subsequent therapy course is administered to said subject until disease progression.

In the context of the present invention, a “therapy course” as mentioned above includes the administration of at least one anti-hematological cancer agent to said subject over a time period optionally with some interval days where no anti-hematological cancer agents are administered to said patient; or optionally with overlapping days of administration. For example, said at least one initial therapy course may include one or more anti-hematological cancer agents administered over a predetermined period of time optionally with interval days between or optionally with overlapping days of administration. Furthermore, in some embodiments said at least one subsequent therapy course may include one or more anti-hematological cancer agents administered over a predetermined period of time optionally with interval days between or optionally with overlapping days of administration.

In some embodiments, said at least one initial therapy course comprises administration of a composition comprising at least one anti-hematological cancer agent.

In further embodiments, said at least one initial therapy course comprises administration of a composition comprising at least one anti-hematological cancer agent that is other than aspacytarabine. (i.e. said composition does not include aspacytarabine). In some embodiments said at least one initial therapy course comprises administration at least one anti-hematological cancer agent wherein said at least one anti-hematological cancer does not include aspacytarabine. In some other embodiments, said at least one initial therapy course comprises administration of at least two anti-hematological cancer agents, one of which comprising anti-hematological cancer agent that is other than aspacytarabine. In some embodiments, this invention provides at least one initial therapy course comprising administering at least two anti-hematological cancer agents, wherein one of the anti-hematological cancer agents is aspacytarabine.

In some embodiments, the methods or regimens described herein comprise at least one initial therapy course, and at least one subsequent therapy course; wherein said at least one initial therapy course is administered until said subject is in remission; and said at least one subsequent therapy course is administered while said subject is in remission.

In another embodiment, the methods or regimens described herein comprise at least one initial therapy course, and at least one subsequent therapy course; wherein said at least one initial therapy course is administered until said subject is in hematological improvement; and said at least one subsequent therapy course is administered while said subject is in hematological improvement.

In another embodiment, the initial therapy course, the subsequent therapy course or both make use of at least one anti-hematological cancer agent. In another embodiments, the anti-hematological cancer agent is aspacytarabine. In another embodiments, the anti-hematological cancer agent is not aspacytarabine and is selected from BCL2 inhibitor, hypomethylation agent (HMA), anti-metabolite, targeted therapy, immune therapy, CAR-T and any combinations thereof. In another embodiment, the anti-hematological cancer agent is a BCL2 inhibitor. In another embodiment, the anti-hematological cancer agent is a hypomethylation agent (HMA). In another embodiment, the anti-hematological cancer agent is an anti-metabolite. In another embodiment, the anti-hematological cancer agent is used as a targeted therapy. In another embodiment, the anti-hematological cancer agent is used as an immune therapy. In another embodiment, the anti-hematological cancer agent is CAR-T.

In another embodiment, BCL2 inhibitor is venetoclax. In another embodiment, hypomethylation agent (HMA) is selected from azacytidine, decitabine, and any combination thereof. In another embodiment, anti-metabolite is selected from cytarabine, aspacytarabine, daunorubicin, idarubicin, cyclosphosphamide, dexamethasone, daunorubicin hydrochloride and cytarabine liposome, and any combination thereof. In another embodiment, targeted therapy is selected from glasdegib, sorafenib, midostaurin, ivosidenib, enasidenib, APR-246, gilteritinib, gemtuzumab ozogamicin, and any combination thereof. In another embodiment, immune therapy is selected from CD47 inhibitor, magrolimab, cusatuzumab, and any combination thereof. In another embodiment, CAR-T is CART-123.

In another embodiments, said at least one anti-hematological cancer agent (which is not aspacytarabine) is selected from: venetoclax, cytarabine, daunorubicin, idarubicin, cyclosphosphamide, dexamethasone, daunorubicin hydrochloride and cytarabine liposome, azacitidine, decitabine, glasdegib, sorafenib, midostaurin, ivosidenib, enasidenib, gemtuzumab ozogamicin, gilteritinib, magrolimab, cusatuzumab, APR-246, CART-123 and any pharmaceutical acceptable salts and/or combinations thereof.

In another embodiment, the anti-hematological cancer agent is venetoclax. In another embodiment, the anti-hematological cancer agent is cytarabine. In another embodiment, the anti-hematological cancer agent is aspacytarabine. In another embodiment, the anti-hematological cancer agent is daunorubicin. In another embodiment, the anti-hematological cancer agent is idarubicin. In another embodiment, the anti-hematological cancer agent is cyclosphosphamide. In another embodiment, the anti-hematological cancer agent is dexamethasone. In another embodiment, the anti-hematological cancer agent is a combination of daunorubicin hydrochloride and cytarabine liposome. In another embodiment, the anti-hematological cancer agent is azacytidine. In another embodiment, the anti-hematological cancer agent is decitabine. In another embodiment, the anti-hematological cancer agent is glasdegib. In another embodiment, the anti-hematological cancer agent is sorafenib. In another embodiment, the anti-hematological cancer agent is midostaurin. In another embodiment, anti-hematological cancer agent is the ivosidenib. In another embodiment, the anti-hematological cancer agent is enasidenib. In another embodiment, the anti-hematological cancer agent is gemtuzumab ozogamicin. In another embodiment, the anti-hematological cancer agent isgilteritinib. In another embodiment, the anti-hematological cancer agent is magrolimab. In another embodiment, the anti-hematological cancer agent is cusatuzumab. In another embodiment, the anti-hematological cancer agent is APR-246. In another embodiment, the anti-hematological cancer agent is CART-123.

In some embodiments, the at least one anti-hematological cancer agent is aspacytarabine.

In some embodiments, the at least one anti-hematological cancer agent is venetoclax, HMA or combination thereof.

In some embodiments, the at least one anti-hematological cancer agent is venetoclax, cytarabine, or combination thereof.

In some embodiments, said at least one anti-hematological cancer agent is at venetoclax or aspacytarabine. In another embodiment, said therapy course (either the initial or the subsequent) comprises a combination therapy of venetoclax and aspacytarabine.

In some embodiments, the initial therapy course comprises administering 5 or 6 days combination of aspacytarabine 4.5 g/m² and venetoclax (50-600/day) and an additional 9-23 days of venetoclax (50-600 mg/day) as the only anti-hematological cancer agent. In another embodiment, the initial therapy course comprising administering 5 or 6 days of combination of aspacytarabine 4.5 g/m² and venetoclax (50-600 mg/day), and additional 9-23 days of venetoclax (50-600 mg/day) as the only anti-hematological cancer agent—is referred to as one course. In another embodiment, the aspacytarabine and venetoclax are given at the same time period or consecutively. In another embodiment, the initial therapy comprises one course. In another embodiment, the initial therapy comprises two courses. In another embodiment, the initial therapy comprises three courses.

In some embodiments, aspacytarabine and at least one anti-hematological cancer agent (which is/are not aspacytarabine) are administered at the same time period or consecutively. In another embodiment, the aspacytarabine is administered at the same time as at least one anti-hematological cancer agent (which is/are not aspacytarabine). In another embodiment, the aspacytarabine is administered before at least one anti-hematological cancer agent (which is/are not aspacytarabine). In another embodiment, the aspacytarabine is administered after at least one anti-hematological cancer agent (which is/are not aspacytarabine). In another embodiment, at least one anti-hematological cancer agent (which is not aspacytarabine) is administered at the same time as aspacytarabine, and for additional period of time without aspacytarabine. In another embodiment, the aspacytarabine is administered at the same time as the at least one anti-hematological cancer agent (which is not aspacytarabine) and for additional period of time without an additional anti-hematological cancer agent (which is/are not aspacytarabine).

In some embodiments, a subsequent therapy course comprises administering aspacytarabine 4.5 g/m². In another embodiment, the subsequent therapy comprises two courses. In another embodiment, the subsequent therapy comprises three courses. In another embodiment, the subsequent therapy comprises up to three courses when aspacytarabine is included.

In another embodiment, the therapy course (either the initial or the subsequent) comprises a combination therapy of venetoclax and azacytidine or combination therapy of venetoclax and cytarabine.

In some embodiments, at least one initial therapy course or/and at least one subsequent therapy course comprises administering aspacytarabine 4.5 g/m²/d (or 2.3 g/m²/d, optional in case of certain toxicities), administered by IV over one hour, once daily for 6 consecutive days.

In another embodiment, the initial or the subsequent course includes 5 days of 1-hour infusion of aspacytarabine.

In some embodiments, aspacytarabine (either administered in the at least one initial therapy course and/or in the at least one subsequent therapy course) is administered in a dose of between 0.3 g/m²/day to 10 g/m²/day. In other embodiments, aspacytarabine (either administered in the at least one initial therapy course and/or in the at least one subsequent therapy course) is administered in a dose of between 1.5 g/m²/day to 6 g/m²/day. In other embodiments, aspacytarabine (either administered in the at least one initial therapy course and/or in the at least one subsequent therapy course) is administered in a dose of between 0.5 g/m²/day to 10 g/m²/day.

In some embodiments, venetoclax (either administered in the at least one initial therapy course and/or in the at least one subsequent therapy course) is administered in a dose of between 50-600 mg/day. In another embodiment, venetoclax is administered in a dose of between 50-150 mg/day. In another embodiment, venetoclax is administered in a dose of between 150-250 mg/day. In another embodiment, venetoclax is administered in a dose of between 250-350 mg/day. In another embodiment, venetoclax is administered in a dose of between 350-450 mg/day. In another embodiment, venetoclax is administered in a dose of between 450-600 mg/day. In another embodiment, venetoclax is administered in a dose of between 50-300 mg/day. In another embodiment, venetoclax is administered in a dose of between 300-600 mg/day.

In some embodiments, venetoclax (either administered in the at least one initial therapy course and/or in the at least one subsequent therapy course) is administered in a dose of between 50-600 mg/day.

In some embodiments, aspacytarabine (administered either in the at least one initial therapy course and/or in the at least one subsequent therapy course) is administered in a dose of at least 1.5 g/m²/day.

In some embodiments, aspacytarabine (administered either in the at least one initial therapy course and/or in the at least one subsequent therapy course) is administered in a dose of at least 2.3 g/m²/day.

In some embodiments, aspacytarabine (administered either in the at least one initial therapy course and/or in the at least one subsequent therapy course) is administered in a dose of at least 4.5 g/m²/day.

In some embodiments, said at least one initial therapy course comprises administration of at least one anti-hematological cancer selected from BCL2 inhibitor, hypomethylation agent (HMA), anti-metabolite, targeted therapy, immune therapy, CAR-T, and any combinations thereof.

In another embodiment, said at least one anti-hematological cancer selected from venetoclax, cytarabine, aspacytarabine, daunorubicin, idarubicin, cyclosphosphamide, dexamethasone, daunorubicin hydrochloride and cytarabine liposome, azacitidine, decitabine, glasdegib, sorafenib, midostaurin, ivosidenib, enasidenib, gemtuzumab ozogamicin, gilteritinib, magrolimab, cusatuzumab, APR-246, CART-123 and any combinations thereof.

In some embodiments, said at least one subsequent therapy course comprises administration of at least one anti-hematological cancer selected from BCL2 inhibitor, hypomethylation agent (HMA), anti-metabolite, targeted therapy, immune therapy, CAR-T, and any combinations thereof. In another embodiments, said at least one anti-hematological cancer selected from venetoclax, cytarabine, aspacytarabine, daunorubicin, idarubicin, cyclosphosphamide, dexamethasone, daunorubicin hydrochloride and cytarabine liposome, azacitidine, decitabine, glasdegib, sorafenib, midostaurin, ivosidenib, enasidenib, gemtuzumab ozogamicin, gilteritinib, magrolimab, cusatuzumab, APR-246, CART-123 and any combinations thereof.

In some embodiments, said treatment of the invention improves at least one clinical condition of said subject selected from duration of response (DOR), survival including overall survival (OS), event-free survival (EFS), leads to negative minimal residual disease (MRD), leads to improved response (e.g. complete remission with incomplete hematological recovery (CRi) to complete remission (CR)), quality of life (QoL), reduces frequency or severity of adverse events, hematological improvement and any combinations thereof.

The invention further provides a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof for use in the treatment of hematological cancer in a subject in need thereof, wherein said treatment comprises: at least one initial therapy course and at least one subsequent therapy course; wherein said at least one initial therapy course is administered until said subject is in remission; and wherein said at least one subsequent therapy course comprises administering said composition while said subject is in remission; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

The invention further provides a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof for use in the treatment of MDS in a subject in need thereof, wherein said treatment comprises: at least one initial therapy course and at least one subsequent therapy course; wherein said at least one initial therapy course is administered until said subject is in hematological improvement; and wherein said at least one subsequent therapy course comprises administering said composition while said subject is in hematological improvement; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

The term “therapeutically effective amount” of the compound is that amount of the compound which is sufficient to provide a beneficial effect to the subject to which the compound is administered. An effective amount of the compound may vary according to factors such as the disease state, age, sex, and weight of the individual.

The terms “treatment”, “treat”, “treating” and the like, are meant to include slowing, arresting or reversing the progression of a disease. These terms also include alleviating, ameliorating, attenuating, eliminating, or reducing one or more symptoms of a disease, even if the disease is not actually eliminated and even if progression of the disease is not itself slowed or reversed. A subject refers to a mammal, preferably a human being.

The term “about” in reference to a numerical value stated herein is to be understood as the stated value +/−10%.

The term “pharmaceutically acceptable salt” of a drug refers to a salt according to IUPAC conventions. Pharmaceutically acceptable salt is an inactive ingredient in a salt form combined with a drug. As used herein, a “pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base.

In another embodiment, the salt is with strong acid such as hydrochloric acid, acetic acid, trifluoroacetic acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid and oxalic acid.

The present invention provides pharmaceutical compositions comprising at least one of the compounds of the present invention and a pharmaceutically acceptable carrier or diluent, optionally further comprising one or more excipients.

The term “pharmaceutically acceptable” means approved by a regulatory agency of the Federal or a state government or listed in the U.S. Pharmacopeia or other generally recognized pharmacopeia for use in animals, and more particularly in humans,

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol, or other synthetic solvents.

For intravenous administration of a therapeutic compound, water is a preferred carrier. Saline solutions and aqueous dextrose and glycerol solutions can also be employed.

The compositions can take the form of solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin.

A composition as used in methods of the invention can further comprise pharmaceutical excipients including, but not limited to, sodium chloride, potassium chloride, magnesium chloride, sodium gluconate, sodium acetate, calcium chloride, sodium lactate, poloxamer and the like. The composition, if desired, can also contain minor amounts of sugar alcohols, wetting or emulsifying agents, and pH adjusting agents.

Pharmaceutical compositions for parenteral administration can also be formulated as suspensions of the active compounds. Such suspensions may be prepared as oily injection suspensions or aqueous injection suspensions. For oily suspension injections, suitable lipophilic solvents or vehicles can be used including fatty oils such as sesame oil, or synthetic fatty acids esters such as ethyl oleate, triglycerides or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds, to allow for the preparation of highly concentrated solutions.

For transmucosal and transdermal administration, penetrants appropriate to the barrier to be permeated may be used in the formulation. Such penetrants, including for example DMSO or polyethylene glycol, are known in the art.

For oral administration, the compounds can be formulated readily by combining the active compounds with pharmaceutically acceptable carriers and excipients well known in the art. Such carriers enable the compounds of the invention to be formulated as tablets, pills, dragees, capsules, liquids, gels, syrups, slurries, suspensions, and the like, for oral ingestion by a subject. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating can be useful if it is desirable to prevent exposure of the compounds of the invention to the gastric environment.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

The dosage of a composition to be administered depends on many factors including the subject being treated, the stage of cancer, the route of administration, and the judgment of the prescribing physician.

A pharmaceutical composition as used in methods of the invention may be administered by any suitable administration route selected from the group consisting of parenteral and oral administration routes. According to some embodiments, the route of administration is via parenteral administration. In various embodiments, the route of administration is intravenous, intraarterial, intramuscular, subcutaneous, intraperitoneal, intracerebral, intracerebroventricular, intrathecal or intradermal administration route. For example, the pharmaceutical compositions can be administered systemically, for example, by intravenous (i.v.) or intraperitoneal (i.p.) injection or infusion. According to a certain embodiment, the pharmaceutical composition is administered by intravenous infusion for 30 minutes to 2 hours, such as for 1 hour. The compositions of the invention may be administered locally and may further comprise an additional active agent and/or excipient.

Toxicity and therapeutic efficacy of the compounds described herein can be determined by standard pharmaceutical procedures in cell cultures or experimental animals, e. g., by determining the IC₅₀ (the concentration which provides 50% inhibition of cell growth) and the MTD (Maximal tolerated dose in tested animals) for a subject compound. The data obtained from cell culture assays and animal studies can be used in formulating a range of dosage for use in human subjects. The exact formulation, route of administration and dosage can be chosen by the individual physician in view of the patient's condition (See e.g., Fingl, et al., 1975, in “The Pharmacological Basis of Therapeutics”, Ch. 1 pp. 1).

According to some embodiments, a pharmaceutical composition used in a method of the invention is administered at least once a month. According to additional embodiments, the pharmaceutical composition is administered at least twice a month. According to further embodiments, the pharmaceutical composition is administered at least once a week. According to yet further embodiments, the pharmaceutical composition is administered at least twice a week. According to still further embodiments, the pharmaceutical composition is administered once a day for at least one week. According to further embodiments, the pharmaceutical composition is administered at least once a day for at least one week or until the subject is cured.

In some embodiments, where the pharmaceutical composition is used for preventing recurrence of cancer, the pharmaceutical composition may be administered regularly for prolonged periods of time according to the clinician's instructions.

In some cases, it may be advantageous to administer a large loading dose followed by periodic (e.g., weekly) maintenance doses over the treatment period. The compounds can also be delivered by slow-release delivery systems, pumps, and other known delivery systems for continuous infusion. Dosing regimens may be varied to provide the desired circulating levels of a particular compound based on its pharmacokinetics. Thus, doses are calculated so that the desired circulating level of a therapeutic agent is maintained.

Typically, the effective dose is determined by the activity and efficacy of the compound and the condition of the subject as well as the body weight or surface area of the subject to be treated. The dose and the dosing regimen are also determined by the existence, nature, and extent of any adverse side effects that accompany the administration of the compounds in a particular subject.

### Regimen

In some embodiments, this invention provides a regimen for treating hematological cancer in a subject in need thereof, wherein said regimen comprises: at least one initial therapy course and at least one subsequent therapy course; wherein said at least one initial therapy course is administered until said subject is in remission; and said at least one subsequent therapy course is administered while said subject is in remission; and wherein the at least one initial therapy course, or the at least one subsequent therapy course or the at least one initial and subsequent therapy course comprises administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

In some embodiments, this invention provides a regimen for treating Myelodysplastic Syndrome (MDS) in a subject in need thereof, wherein said regimen comprises: at least one initial therapy course and at least one subsequent therapy course; wherein said at least one initial therapy course is administered until said subject is in hematological improvement; and said at least one subsequent therapy course is administered while said subject is in hematological improvement; and wherein the at least one initial therapy course, or the at least one subsequent therapy course or the at least one initial and subsequent therapy course comprises administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

In some embodiments the methods described herein refer to regimens of treatment. The methods and regimens described herein are used herein interchangeably.

The following examples are to be considered merely as illustrative and non-limiting in nature. It will be apparent to one skilled in the art to which the present invention pertains that many modifications, permutations, and variations may be made without departing from the scope of the invention.

### EXAMPLES

Example 1: Randomized, Open-Label Study to Comparing, the Efficacy of Aspacytarabine Consolidation Therapy, to Standard of Care Therapy in Subjects with Acute Myeloid Leukemia in First Remission Following Treatments with Venetoclax in Combination with Azacitidine

Standard of care (SOC) (Treatment options post randomization):
Venetoclax+azacitidine administered monthly until relapse or sever toxicity.

Study population: Adult subjects with newly diagnosed acute myeloid leukemia (AML) who entered a first remission, with or without count recovery, following initial treatment of venetoclax and azacitidine.

Study Objective: Primary objective-event free survival (EFS), duration of response (DOR), and overall survival (OS) in the aspacytarabine treated subjects compared to current standard of care. Secondary objectives-compliance, and quality of life in subjects treated with aspacytarabine compared standard of care. Conversion from minimal residual disease (MRD) positive to negative.

Study Design: Prospective open label, multi-center, randomized, parallel-group study to compare the effect of aspacytarabine to standard of care in AML subjects in first remission. Subjects will be randomly assigned with equal probability to 1 of the 2 treatment groups: (1) Aspacytarabine 4.5 g/m²/d (or 2.3 g/m²/d, optional in case of certain toxicities), administered IV over one hour, once daily for 5 or 6 consecutive days. and (2) Standard of care (SOC) administration of venetoclax+azacitidine.

Subjects in the aspacytarabine group will be treated with 2-4 subsequent (in-remission) courses. Each course includes 5 or 6 days of 1-hour infusion of aspacytarabine.

After the last aspacytarabine consolidation course the subjects will be followed by in clinic visits for the length of the study.

Subject in the SOC group will be treated monthly and followed according to the SOC.

### Example 2—Dose Escalation and Expansion Study with Aspacytarabine and Venetoclax in Patients with Newly Diagnosed Acute Myeloid Leukemia Unfit for Intensive Induction Therapy

Study population: Adult subjects with newly diagnosed acute myeloid leukemia (AML) unfit for induction (initial) therapy

Study Objective: Define the maximal tolerated dose (MTD) of venetoclax when given in combination with aspacytarabine, followed by efficacy, pharmacokinetics, and safety in unfit patients with newly diagnosed AML.

Study Design: This is a dose escalation and expansion, open-label, multi-center study with BST-236 in combination with venetoclax, in adult subjects with newly diagnosed AML unfit for induction therapy. Part 1 of the study will identify the maximal tolerated dose of this combination. In the second part aspacytarabine will be administered with the chosen dose of venetoclax. Patients in complete response will be treated with up to 3 courses of aspacytarabine 4.5 g/m2 or less in case of earlier toxicity. Following the last aspacytarabine course subjects will be followed by in clinic visits for the rest of the study.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating hematological cancer in a subject in need thereof, wherein said method comprises: at least one initial therapy course and at least one subsequent therapy course; wherein said at least one initial therapy course is administered until said subject is in remission; and said at least one subsequent therapy course is administered while said subject is in remission; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.

2. The method of claim 1, wherein said at least one initial therapy course comprises administering at least one anti-hematological cancer agent or at least two anti-hematological cancer agents.

3. The method of claim 2, wherein said at least one initial therapy course comprises administering aspacytarabine.

4. (canceled)

5. (canceled)

6. The method of claim 2, wherein said at least one initial therapy course comprises administering at least one anti-hematological cancer agent that is not aspacytarabine.

7. (canceled)

8. The method of claim 1, wherein said at least one subsequent therapy course comprises administering aspacytarabine or at least one anti-hematological cancer agent that is not aspacytarabine.

9. The method of claim 1, wherein said at least one subsequent therapy course comprises administering at least two anti-hematological cancer agents, wherein one of said anti-hematological cancer agent is aspacytarabine, wherein at least one of said anti-hematological cancer agent is not aspacytarabine or a combination thereof.

10. (canceled)

11. (canceled)

12. The method of claim 1, wherein said at least one initial therapy course comprises administering aspacytarabine and at least one additional anti-hematological cancer agent; and wherein said at least one subsequent therapy course comprises administering aspacytarabine and at least one additional anti-hematological cancer agent; wherein the at least one additional anti-hematological cancer agent in the initial and subsequent therapy is the same or different.

13. The method of claim 1, wherein said at least one initial therapy course comprises administering aspacytarabine and at least one additional anti-hematological cancer agent; and wherein said at least one subsequent therapy course comprises aspacytarabine.

14. The method of claim 1, wherein said at least one initial therapy comprises administering at least one anti-hematological cancer agent other than aspacytarabine; and wherein said at least one subsequent therapy course comprises aspacytarabine and optionally at least one additional anti-hematological cancer agent.

15. (canceled)

16. The method of claim 1 wherein the at least one initial therapy comprises number of courses until remission, wherein each course is the same or different, wherein the at least one subsequent therapy comprises number of courses, wherein each course is the same or different, or any combination thereof.

17. (canceled)

18. The method of claim 1, wherein said hematological cancer is selected from Leukemia, Lymphoma, Multiple Myeloma, Myelodysplastic Syndrome (MDS), myeloproliferative neoplasm (MPN) and any combinations thereof.

19. The method of claim 18, wherein said Leukemia is selected from Acute Myeloid Leukemia (AML), Acute Lymphoblastic Leukemia (ALL), Chronic Myeloid Leukemia (CML), Chronic Lymphoblastic Leukemia (CLL), and any combinations thereof, wherein said Lymphoma is selected from Hodgkin's Lymphoma, Non-Hodgkin's Lymphoma (NHL), and any combinations thereof.

20. (canceled)

21. The method of claim 2, wherein said at least one anti-hematological cancer agent is selected from BCL2 inhibitor, hypomethylation agent (HMA), anti-metabolite, targeted therapy, immune therapy, CAR-T, and any combinations thereof.

22. The method of claim 2, wherein said at least one anti-hematological cancer agent is selected from: venetoclax, cytarabine, aspacytarabine, daunorubicin, idarubicin, cyclosphosphamide, dexamethasone, daunorubicin hydrochloride and cytarabine liposome, azacitidine, decitabine, glasdegib, sorafenib, midostaurin, ivosidenib, enasidenib, gemtuzumab ozogamicin, gilteritinib, magrolimab, cusatuzumab, CD47 inhibitor, APR-246, CART-123 and any combinations thereof.

23. The method of claim 2, wherein said at least one anti-hematological cancer agent is at least one of venetoclax and hypomethylation agent (HMA), at least one of Venetoclax and cytarabine or at least one of venetoclax and aspacytarabine.

24. (canceled)

25. (canceled)

26. The method of claim 1, wherein aspacytarabine is administered in a dose of between 0.3 g/m²/day to 10 g/m²/day.

27.-29 (canceled)

30. The method of claim 1 wherein said at least one subsequent therapy course comprises administering at least one anti-hematological cancer agent selected from: venetoclax, cytarabine, aspacytarabine, daunorubicin, idarubicin, cyclosphosphamide, dexamethasone, daunorubicin hydrochloride and cytarabine liposome, azacitidine, decitabine, glasdegib, sorafenib, midostaurin, ivosidenib, enasidenib, gemtuzumab ozogamicin, gilteritinib, cusatuzumab, APR-246, CART-123 and any combinations thereof.

31. The method of claim 1, wherein the treatment improves at least one clinical condition of said subject selected from DOR (duration of response), survival including overall survival (OS), event-free survival (EFS), Minimal residual disease (MRD), leads to improved response, QoL, frequency or severity of adverse events, hematological improvement and any combinations thereof.

32. A composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof for use in the treatment of hematological cancer in a subject in need thereof, wherein said treatment comprises: at least one initial therapy course and at least one subsequent therapy course; wherein said at least one initial therapy course is administered until said subject is in remission; and wherein said at least one subsequent therapy course comprises administering said composition while said subject is in remission; and wherein the at least one initial therapy course, or the at least one subsequent therapy course or both therapy courses comprise administering a composition comprising (2S)-2-amino-4-[[1-[(2R,3S,4S,5R)-3,4-dihydroxy-5-(hydroxymethyl)oxolan-2-yl]-2-oxopyrimidin-4-yl]amino]-4-oxobutanoic acid (aspacytarabine) or a pharmaceutically acceptable salt thereof.
